(19) United States
(12) Patent Application Publication
(10) Pub. No.: US 2013/0225472 A1
(43) Pub. Date: Aug. 29, 2013

(54) SOLID COMPOSITIONS CONTAINING GLYCOL ETHER AND WATER

(71) Applicants: Lisa Wu, Shanghai (CN); Mac Lai, Shanghai (CN); Yin Zhu, Shanghai (CN)

(72) Inventors: Lisa Wu, Shanghai (CN); Mac Lai, Shanghai (CN); Yin Zhu, Shanghai (CN)

(73) Assignee: JOHNSON & JOHNSON CONSUMER COMPANIES, INC., Skillman, NJ (US)

(21) Appl. No.: 13/818,746

(22) PCT Filed: May 3, 2012
(86) PCT No.: PCT/US12/36332

§ 371 Date: May 6, 2013

(30) Foreign Application Priority Data
May 6, 2011 (CN) 201110127008.0

(51) Int. Cl. A61K 8/02; A61Q 5/02; A61Q 19/10

(52) U.S. Cl. CPC A61K 8/0216; A61Q 19/10; A61Q 5/02
USPC 510/447; 510/506; 510/470; 510/473

(57) ABSTRACT

Provided are solid compositions comprising a glycol ether, a gelling agent, and water. Also provided are methods of making such compositions.

### FIELD OF INVENTION

This invention relates to solid compositions and methods of making such. More specifically, it relates to solid compositions, including but not limited to solid cleansing compositions, that comprise relatively high amounts water and exhibit relatively low mush and cracking.

### DESCRIPTION OF RELATED ART

A wide variety of solid compositions, made in a variety of ways, for consumer use are known in the art. For example, a variety of solid compositions are made by gelling organic liquids using gelling/solidifying agents such as dibenzylidene sorbitol (also called dibenzaldehyde monosorbitol acetal, or dibenzyl monosorbitol acetal or dibenzylidene monosorbitol acetal) and derivatives thereof (such as those which are substituted on one or both of the aromatic rings with a fluorine or methoxy group and those which have the sorbitol portion replaced with other reduced sugars such as xylitol or ribitol, as described, for example, in U.S. Pat. No. 5,609,855.)

However, applicants have recognized that many cosmetic products, especially rinse-off products, tend to contain a relative medium to high dosage of water, and that in attempts to make solid cosmetic products, e.g. solid shampoos, solid shower gels, or solid cleansing bars, the inclusion of a large amount of water or water-based dispersion into an oil gelling composition comprising dibenzylidene sorbitol or a derivative thereof may cause phase separation or inability for the composition to solidify. In addition, applicants have recognized that a variety of known solid compositions comprising dibenzylidene sorbitol or a derivative thereof tend to have undesirable properties, such as relatively high mush and crack, associated therewith.

For example, a clear cleansing bar made with dibenzylidene sorbitol, a glycol solvent, and surfactants in the relative absence of water is described in U.S. Pat. No. 6,514,919 to Lambino et al. While Lambino indicates that the clear cleansing bar therein tends to have relatively low mush, applicants have recognized the desirability of incorporating water in, and/or further reducing the mush and/or cracking associated with, solid compositions including cleansers.

Another example includes U.S. Pat. No. 7,045,491 to Hourigan which describes a non-clear cleansing bar made with anionic soap, dibenzylidene sorbitol/xylitol/ribitol, glycerin and water. Applicants have recognized however, that such bars may tend to exhibit undesirable mush and/or cracking properties for certain applications and tend to lack clarity.

Accordingly, applicants have recognized the need for solid compositions comprising water that tend to exhibit improved mush and/or cracking properties, and in certain embodiments further exhibit clarity and/or mildness properties.

### SUMMARY OF THE INVENTION

In one aspect, the present invention provides a solid composition comprising a glycol ether, a gelling agent, and water.

In another aspect, the present invention provides a method of making a solid composition of the present invention.

Applicants have discovered unexpectedly that solid compositions of the present invention comprising a glycol ether solvent in combination with a gelling agent, and water tend exhibit improved properties such as mush and hardness as compared to other comparable solid compositions. In addition, applicants have discovered that in certain embodiments, the present compositions allow for the incorporation of relatively medium to high levels of water while still solidifying and exhibiting beneficial mush, hardness, and/or transparency properties, whereas other comparable compositions do not solidify or do not exhibit the beneficial combination of properties at such relatively medium to high levels of water.

For example, as shown in the Examples and described herein, applicants have measured the mush and crack associated the compositions of the present invention as compared to comparable compositions made via the descriptions of U.S. Pat. No. 6,514,919 to Lambino et al. and U.S. Pat. No. 7,045,491 to Hourigan. While the solid compositions from Lambino and Hourigan exhibited some, to significant, mush and/or crack, the compositions of the present invention exhibited essentially no mush and no crack. In addition, as shown in the Examples, compositions of the present invention tended to solidify with the incorporation of relative high levels of water, whereas comparable compositions tended to not solidify at such levels of water. Also as shown, certain compositions of the present invention tended to be transparent.

The solid compositions of the present invention are also beneficial in that they provide ease of transportation and are portable for use while travelling or participating in outdoor activities such as camping. They also tend to be economical as no complicated packaging is required, and they often require reduced levels of preservatives as compared to conventional liquid formulations.

### DESCRIPTION OF THE INVENTION

As used herein, a “solid” composition refers to a composition that is firm enough at ambient temperature (e.g. room temperature, about 27° C. at 50% relative humidity) that it can be handled by hand pressure and removed from packaging without observable softness or change in shape during the process. Preferably, a solid composition has a hardness of about 3N or greater.

According to certain embodiments, the present invention provides solid compositions comprising a glycol ether, a gelling agent, and water.

As used herein, unless otherwise specified, all percentages of ingredients in compositions are weight percent of active/solid ingredient based on the total weight of composition.

Any suitable glycol ether may be used in accord with the present invention. Examples of suitable glycol ethers include, but are not limited to, ethylene glycol alkyl ethers, such as, ethylene glycol monomethyl ether, ethylene glycol monoethyl ether, ethylene glycol monopropyl ether, ethylene glycol monoisopropyl ether, ethylene glycol monobutyl ether and the like, ethylene glycol aryl ethers, such as, ethylene glycol monophenyl ether, ethylene glycol monobenzyl ether and the like, ethylene glycol dialkyl ethers, such as ethylene glycol dimethyl ether, ethylene glycol diethyl ether, ethylene glycol dibutyl ether and the like, diethylene glycol alkyl ethers, such as, diethylene glycol monomethyl ether, diethylene glycol monoethyl ether, diethylene glycol mono-n-butyl ether and the like, dipropylene glycol alkyl ethers, such as, dipropylene glycol methyl ether, propylene glycol n-propyl ether, dipropylene glycol n-propyl ether and the like, and tripropylene glycol alkyl ethers, such as, tripropylene glycol methyl ether and the like, as well as mixtures of any two or more of such glycol ethers and the like. Certain preferred examples of glycol ethers include tripropylene glycol methyl ether, dipropylene glycol methyl ether, and the like. In certain particularly preferred embodiments, the glycol ether comprises or is tripropylene glycol methyl ether.

Any suitable amounts of glycol ether may be used in accord with the present invention. In certain embodiments, the compositions comprise from greater than zero to about 80% by weight of the composition of glycol ether. In more preferred embodiments, the compositions comprise from about 5% to about 75%, including, for example in certain embodiments, from about 5% to about 30%, and from about 25 to about 60% of glycol ether.

Any of variety of materials that can dissolve in a liquid system for use in making a solid cleanser of the present invention and then contribute to solidifying the liquid system, or forming a gel-like solid from the liquid system may be used as a gelling agent in accord with the present invention. Examples of suitable gelling agents include, but are not limited to, metal (anionic) soaps, for example, sodium, potassium, lithium, ammonium, or hydroxyethyl ammonium salts of C8 (or higher) alkyl fatty acids, including C12-C₁₋₈ alkyl fatty acids such as sodium or lithium stearate and the like; 12-hydroxystearic acid; waxes including but not limited to paraffin wax, ceresin wax, microcrystalline wax, Fischer-Tropsch wax, polyethylene wax, carnauba wax, and candelila wax; dextrin fatty acid esters including but not limited to dextrin palmitate; sucrose fatty acid esters; anhydrous silicic acids; organic modified clay minerals; glyceryl behenate/eicosadioate; dibenzylidene alditols including but not limited to dibenzylidene alditols such as dibenzylidene sorbitol, dibenzylidene xylitol, dibenzylidene ribitol, 4-methyl dibenzylidene sorbitol, 4-ethyl dibenzylidene sorbitol, 4-chloro dibenzylidene sorbitol, 2,4-dimethyl dibenzylidene sorbitol, bis(3,4-dimethylesbenzylidene) sorbitol acetal, tribenzylidene sorbitol; or amino acid based oil gelling agents including but not limited to dibutyl ethlhexanoyl glutamide, GP-1 (dibutyl lauroyl glutamide). Examples of preferred gelling agents include dibenzylidene alditols such as dibenzylidene sorbitol, dibenzylidene xylitol, dibenzylidene ribitol, 4-methyl dibenzylidene sorbitol, 4-ethyl dibenzylidene sorbitol, 4-chloro dibenzylidene sorbitol, 2,4-dimethyl dibenzylidene sorbitol, bis(3,4-dimethylesbenzylidene) sorbitol acetal, tribenzylidene sorbitol and mixtures of two or more thereof. In certain particularly preferred embodiments, the gelling agent comprises or is dibenzylidene sorbitol.

Any suitable amounts of gelling agent may be used in accord with the present invention. In certain embodiments, the compositions comprise from greater than zero to about 10% by weight of the composition of gelling agent. In more preferred embodiments, the compositions comprise from about 0.5% to about 5%, more preferably from about 0.5 to about 2% by weight of gelling agent.

While, as indicated above, the compositions of the present invention may comprise metal soaps in accord with certain embodiments of the present invention, according to certain preferred embodiments, the compositions of the present invention are essentially free of metal soaps. As used herein, a composition that is “essentially free of metal soaps” means the composition that has less than 2% of that ingredient by weight based on the total weight of the composition. Preferably, a composition that is essentially free of metal soap has about 1% or less, more preferably about 0.5% or less, more preferably about 0.1% or less, more preferably about 0.01% or less by weight based on the total weight of composition of metal soaps. In certain particularly preferred embodiments, a composition that is essentially free of metal soaps has no metal soap therein.

The compositions of the present invention preferably comprise water in an amount of about 10% or greater. In certain preferred embodiments, the compositions comprise from about 10% to about 70% of water, more preferably from about 10% to about 60% of water. In certain more preferred embodiments, the compositions comprise from about 20% to about 60% of water, including from about 30% to about 60% of water. In certain other more preferred embodiments, the compositions comprise at least 35% by weight of water, including from 35% to about 70% and from 40% to about 70% by weight of water.

According to certain preferred embodiments, the compositions of the present invention further comprise a solidifying synergist. Any of variety of materials that, in the presence of a gelling agent in a liquid system in accord with the present invention, can also contribute to solidifying the liquid system or forming a gel-like solid from the liquid system, may be used as a solidifying synergist in accord with the present invention. In certain embodiments, by adding the solidifying synergists to a liquid system with a gelling agent, the resultant gel strength or the gelling point of the system tends to be increased. Examples of solidifying synergists include but are not limited to cellulose and guar derivatives including but not limited to hydroxypropyl cellulose, and the like; acrylic acid polymers, polyacrylamides, alkylene/alkylene oxide polymers, polyethylene glycol esters, such as PEG-150 distearate, PEG-120 methyl glucose dioleater, and the like, smectite hydrophilic and organoclays, hydrated and fumed silicas, gelatin, keratin, xanthan and guar gums, carrageenan, agar and alginates. According to certain preferred embodiments, the solidifying synergist comprises or is hydroxypropyl methyl cellulose.

Any suitable amounts of solidifying synergists may be used in accord with the present invention. In certain embodiments, the compositions comprise from greater than zero to about 10% by weight of the composition of solidifying synergists. In more preferred embodiments, the compositions comprise from about 0.05% to about 5%, more preferably from about 0.5 to about 2% by weight of solidifying synergists.

According to certain embodiments, the compositions of the present invention optionally comprise a co-solvent. Suitable co-solvents include, but are not limited to, dihydroxy aliphatic alcohols containing from about 3 to 6 carbon atoms, such as 1,3 propylene glycol, 1,3-butylene glycol, 1,4 butylene glycol and hexylene glycol; polyethylene and polypropylene glycols, such as dipropylene glycol, tripropylene glycol, tetrapropylene glycol and 1,3-propanediol; monohydric alcohols, such as ethanol and propanol; polyhydric alcohols or polyols, such as maltitol, xylitol, sorbitol, glycerin, pentaerythritol, glycerol, diglycerol and polyglycerol, ethylene glycol; and mixtures thereof. Preferably, the co-solvent is a polyol, more preferably a polyol selected from the group consisting of propylene glycol, glycerin, 1,3-butylene glycol, sorbitol, and combinations of two or more thereof. In certain more preferred embodiments, the co-solvent comprises or is propylene glycol and/or 1,3-butylene glycol.

Any suitable amounts of co-solvents may be used in accord with the present invention. In certain embodiments, the compositions comprise from zero to about 60% by weight of the composition of co-solvents. In more preferred embodiments, the compositions comprise from about 0% to about 50%, for example from about 5 to about 30% or 20% to about 50% by weight of the composition of co-solvents.

While glycerin may be a suitable co-solvent in accord with certain embodiments of the present invention, according to certain preferred embodiments, the compositions of the present invention are essentially free of glycerin. As used herein, a composition that is “essentially free of glycerin” means the composition that has less than 2% of that ingredient by weight based on the total weight of the composition. Preferably, a composition that is essentially free of glycerin has about 1% or less, more preferably about 0.5% or less by weight based on the total weight of composition of glycerin. In certain particularly preferred embodiments, a composition that is essentially free of glycerin has no glycerin therein.

In certain preferred embodiments, the total weight percent of glycol ether and any optional co-solvents combined is from greater than zero to about 80% by weight in the composition. In certain more preferred embodiments, the compositions of the present invention comprise from about 5 to about 70% by weight, and more preferably from about 20 to about 60% by weight of combined glycol ether and optional co-solvents.

In certain preferred embodiments, for example in embodiments wherein the solid composition is a solid cleanser, the solid compositions of the present invention further comprise one or more surfactants. Any of a variety of surfactants selected from a group consisting of anionic, amphoteric, non-ionic, cationic surfactants, and combinations of two or more thereof may be used in accord with the present invention. Surfactants may be in solid form, such as granules of surfactant, surfactant powder, and the like, or liquids. In certain preferred embodiments, the surfactants are liquid surfactants.

As used herein, the term “anionic surfactant” refers to an ionic surfactant in which the hydrophilic portion of the surfactant carries negative charge. A description of anionic surfactants can be found in numerous texts and monographs, such as Rieger, Surfactant Encyclopedia, 2^{nd} Ed., C&T Ingredient Resource Series of COSMETICS AND TOILETRIES® magazine, published by Allured Publishing Corporation, Carol Stream, Ill. (1996), the relevant disclosures of which are incorporated by reference. Examples of suitable anionic surfactants include acylamino acids (and salts), such as acylgutamates, acyl peptides, sarcosinates, taurates, and the like; carboxylic acids (and salts), such as alkanoic acids (and alkanoates), ester carboxylic acids, ether carboxylic acids, and the like; phosphoric acid esters (and salts); sulfonic acids (and salts), such as acyl isethionates, alkyl isethionates, alkylaryl sulfonates, alkyl sulfonates, alkyl sulfosuccinates (and salts), and the like; and sulfuric acid esters, such as alkyl ether sulfates, alkyl sulfates, and the like. Examples of certain preferred anionic surfactants include alkyl sulfates; alkyl ether sulfates; alkyl monoglyceryl ether sulfates; alkyl monoglyceride sulfates; alkyl monoglyceride sulfonates; alkyl sulfonates; alkylaryl sulfonates; alkyl sulfosuccinates; alkyl ether sulfosuccinates; alkyl sulfosuccinamates; alkyl amidosulfosuccinates; alkyl carboxylates; alkyl amidoethercarboxylates; alkyl succinates; fatty acyl sarcosinates; fatty acyl amino acids; fatty acyl taurates; fatty alkyl sulfoacetates; alkyl phosphates, alkyl and acyl isethionates; and mixtures of two or more thereof, wherein the alkyl group has from about 10 to about 16 carbon atoms. In certain other preferred embodiments, the anionic surfactant comprises one or more alkyl sulfates, alkyl ether sulfates, alkyl isethionates, alkyl sulfonates, alkyl ether sulfonates, alkyl sulfosuccinates, alkyl ether sulfosuccinates, and combinations of two or more thereof. In certain particularly preferred embodiments the anionic surfactant comprises sodium laureth sulfate, sodium cocoylisethionate, sodium C 14-16 olefin sulfonate, disodium laureth sulfosuccinate, or a combinations of two or more thereof. In certain more preferred embodiments, the anionic surfactant comprises or is sodium C14-16 olefin sulfonate, disodium laureth sulfosuccinate, or a mixture of two or more thereof.

Any suitable amounts of anionic surfactant may be used in accord with the present invention. In certain embodiments, the compositions comprise from greater than zero to about 30% by weight of the composition of anionic surfactant. In more preferred embodiments, the compositions comprise from about 0.5% to about 30%, more preferably from about 1 to about 25% by weight of anionic surfactant, more preferably from about 5 to about 15% by weight of active anionic surfactant.

As used herein, the term “amphoteric” means: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions. Examples of amphoteric surfactants suitable for use in the present invention include, but are not limited to amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures of two or more thereof. Examples of certain preferred amphoteric surfactants include alkyl betaines, amidoalkylbetaines, phosophobetaines, alkylamphoglycinates, and combinations of two or more thereof. In certain particularly preferred embodiments, the amphoteric surfactant is selected from the group consisting of cocamidopropylbetaine, lauroamphoglycinate, lauric-myristic phosphobetaines, lauryl betaine, and combinations of two or more thereof, and even more preferably from sodium lauroamphoacetate, disodium cocamidopropylbetaine and mixtures thereof.

Any suitable amounts of amphoteric surfactant may be used in accord with the present invention. In certain embodiments, the compositions comprise from greater than zero to about 30% by weight of the composition of amphoteric surfactant. In more preferred embodiments, the compositions comprise from about 0.5% to about 30%, more preferably from about 1 to about 20% by weight of amphoteric surfactant, more preferably from about 2 to about 20% by weight of active amphoteric surfactant.

As used herein, the term “nonionic surfactant” refers to an ionic surfactant in which the hydrophilic portion of the surfactant carries no charge. One class of nonionic surfactants useful in the present invention are polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester. Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl glucosides have about 1 to about 6 glucose residues per molecule of alkyl glucoside. Examples of certain preferred nonionic surfactants include PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the trade name, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the trade name “Tween 20”.

Any suitable amounts of nonionic surfactant may be used in accord with the present invention. In certain embodiments, the compositions comprise from greater than zero to about 30% by weight of the composition of nonionic surfactant. In more preferred embodiments, the compositions comprise from about 0.5% to about 30%, more preferably from about 1 to about 20% by weight of active nonionic surfactant.

As used herein, the term “cationic surfactant” refers to an ionic surfactant in which the hydrophilic portion of the surfactant carries positive charge. Useful cationic surfactants include N-alkyl betaines, quaternary ammonium compounds, amido-amines, N-alkylamines, N-alkylamine oxides, amido-amine betaines, amido-amine salts, amido-amine oxides, sultaines and ethoxylated amines.

Any suitable amounts of cationic surfactant may be used in accord with the present invention. In certain embodiments, the compositions comprise from greater than zero to about 15% by weight of the composition of cationic surfactant. In more preferred embodiments, the compositions comprise from about 0.5% to about 15%, more preferably from about 1 to about 10% by weight of active cationic surfactant.

The composition of the present invention may have any suitable pH associated therewith. According to certain preferred embodiments, the compositions of the present invention exhibit a pH value, measured as described below, of about 8 or less, more preferably about 7 or less, more preferably less than 7.

Optional other ingredients may be incorporated into the composition of this invention. Examples of such ingredients include active agents for a variety of end benefits, antioxidants, perfumes, colorants and dyes, beads, antimicrobial agents, preservatives, and humectants such as sugar, urea, and the like.

The compositions of the present invention are solid compositions that may take any suitable form. For example, the compositions may be in the form of traditional solid materials, such as, bars, sticks, cakes, and the like, in any of a variety of shapes (conventional and novelty), and may also be in the form of gels and gel-like materials. In certain preferred embodiments, the compositions are cleansing bars.

According to certain preferred embodiments, the solid compositions of the present invention exhibit relatively low mush. In particular, as measured in accord with the Mush Test Method described herein below, certain preferred compositions of the present exhibit a Total Mush of about 10 g/50 cm² or less, more preferably about 5 g/50 cm² or less, even more preferably about 2 g/50 cm² or less, even more preferably about 1 g/50 cm² or less, even more preferably about 0.5 g/50 cm² or less, and even more preferably essentially no mush.

According to certain preferred embodiments, the solid compositions of the present invention exhibit relatively low to no cracking. In particular, as measured in accord with the Crack Test Method described herein below, certain preferred compositions of the present exhibit crack Grade I or essentially no crack, and even more preferable essentially no crack.

Applicants have recognized that according to certain embodiments, the compositions of the present invention are suitable for making translucent or transparent compositions. As used herein, the term “transparent” refers to a solid cleanser composition which has a maximum transmittance of light therethrough, as measured in accord with the Transparency Test described hereinbelow, of at least 35%, preferably about 50% or greater. The term “translucent” refers to a solid cleanser composition which has a maximum transmittance of light therethrough, as measured in accord with the Transparency Test described hereinbelow, of greater than 2% but less than 35%. In certain preferred embodiments, the compositions of the present invention are transparent.

According to certain preferred embodiments, the solid compositions of the present invention exhibit a desirable hardness. In particular, as measured in accord with the Hardness Test Method described herein below, certain preferred compositions of the present exhibit a Hardness value of from about 3 to about 50 Newtons (N), more preferably from about 10 to about 50 N.

According to certain preferred embodiments, the solid cleansing compositions of the present invention exhibit a desirable foam volume. In particular, as measured in accord with the Foaming Test Method described herein below, certain preferred compositions of the present exhibit a Foam Volume of about 5 cm or greater. In certain more preferred embodiments, compositions of the present exhibit a Foam Volume of about 8 cm or greater, more preferably about 14 or greater, and even more preferably about 18 or greater.

The present invention further provides methods of making a solid composition of the present invention. According to certain embodiments, the methods of the present invention comprise combining a heated gelling mixture comprising one or more glycol ethers and gelling agents, and optionally one or more solidifying synergists and optional co-solvents, as described above, with an aqueous liquid, optionally comprising one or more surfactants, and cooling to form a solid composition of the claimed invention.

In certain preferred embodiments, the solid compositions are prepared by mixing a glycol ether and optional co-solvent and heating to at least about 80° C., then adding the gelling agent and solidifying synergists and continuing to heat to about 110° C. to about 140° C., mixing till a clear mucilage is formed. To this heated mixture is added a solution of surfactants and water. The composition is mixed until homogenous at about 80° C. to about 90° C. Optional ingredients like perfume and colorants are added when temperature reaches below about 90° C. The molten stock is then poured into suitable molds of different forms made of plastic or rubber and allowed to cool and harden at ambient conditions.

In certain embodiments, the solid cleanser compositions of the present invention are prepared by solidifying commercially available liquid cleansers, such as liquid shower gels and shampoos. The water content of conventional liquid shower gels and shampoos is often about 70% or greater by weight of the composition. This aqueous phase can be solidified using the gelling system of the present invention. The commercial liquid shower gel or shampoo is added into a heated gelling mixture comprising glycol ether and gelling agent, and the mixing is continued to obtain a homogenous composition. This homogenous composition is then poured into suitable molds of different forms, such as those made of plastic or rubber and allowed to cool and harden at ambient conditions.

### EXAMPLES

The following examples will more fully illustrate the embodiments of this invention. All parts, percentages and proportions referred to herein are by weight unless otherwise indicated. The examples are provided for illustrative purposes and should not be considered as limiting the scope of the invention.

The sources for the materials utilized in the following examples were as follows: Dibenzylidene sorbitol was obtained from Milliken & Company under the tradename MILLITHIX® 925S; Hydroxypropyl methyl cellulose was obtained from DOW under the tradename METHOCEL K100 Premium; Tripropylene glycol methyl ether was obtained from DOW under the tradename DOWANOL TPM GLYCOL ETHER; Dipropylene glycol methyl ether was obtained from Polystar under the tradename DIPROPYLENE GLYCOL METHYL ETHER; Propylene glycol was obtained from Dongying Hi-Tech under the tradename Propylene Glycol USP; Glycerin was obtained from Kerry Oleochemical under the tradename RG-995(USP); Cocamidopropyl Betaine was obtained from Evonik Industries under the tradename TEGO® Betain F 50J (44% water); Coco-glucoside was obtained from Cognis under the tradename Plantacare 818 UP (50% water); Urea was obtained from Shanghai Reagent co. under the tradename UREA; Sodium laureth sulfate (2E0) was obtained from Cognis under the tradename TEXAPON N70 LS-J (30% water); Sodium C14-16 Olefin sulfonate was obtained from Zhongqing Chemical under the tradename AOS 35 (63% water); Sodium lauroamphoacetate was obtained from Cognis under the tradename DEHYTON KE 1889/50 (50% water); Cocamide MEA was obtained from Cognis under the tradename Comperlan 100C; Sodium lauroyl glutamate was obtained from Ajinomoto co. inc under the tradename AMISOFT LS-11 (surfactant powder); Sodium cocoyl isethionate was obtained from Clariant under the tradename HOSTAPON SCI 85 (surfactant powder); Sodium cocoyl glutamate was obtained from Ajinomoto co. inc under the tradename AMISOFT CS-11 (surfactant powder); Disodium Laureth Sulfosuccinate was obtained from EOC GROUP under the tradename Euranaat LS3 (60% water); PEG-150 distearate was obtained from Rohm & Haas under the tradename Aculyn-60, PEG-120 Methyl Glucose Dioleate was obtained from Lubrizol under the tradename DOE-120; Gellan gum was obtained from CP Kelco US, Inc. under the tradename KELCOGEL CG-HA; and Glyceryl oleate citrate was obtained from Symrise under the tradename Dracorin GOC. Water used is dionized (DI) water.

The test methods used in the following examples and in accord with the present invention were as follows:

Mush Test Method:

A test solid (soap bar) was cut into an approximately rectangular box shape. The bar was weighed and soaked in DI water at 20° C.±1° C. for 2 hours. The increase in weight was recorded and the mush gently removed by carefully scraping the bar with a plastic scraper. The scraped bars were wiped with a soft tissue to remove the last traces of mush. The remaining portion of the test solid was allowed to dry for 24 hours at 22-25° C., 60-70% humidity, and then weighed. The weight of mush is calculated as the original weight of the test solid minus the weight of the remaining portion after drying. The surface area of the test solid, or that portion of the test solid immersed in water, is calculated in square cm (cm²). Total Mush is then calculated as the weight of mush (g) per 50 cm² of surface area (i.e. g/50 cm²).

Crack Test Method:

Rub soap bar with twisting motion under in running water for 1 minute to rub the outer layer of the surface. A straightened paper clip is inserted into the top portion of the soap bar and connected at the other end to a cord and an iron frame to suspend the bar in a soaking pan. DI Water at approximately 21° C. is poured into the pan to submerge ⅔ of the soap bar in the water. The water should not touch the clips inserted into the top portion of the bar. The bar should not touch the sides or bottom of the pan, nor other bars, if any. The bar is soaked for 6 hours. After 6 hours the bar is removed from the water and hung for 24 hours to dry at 22-25° C., 60-70% humidity. Then pictures of the bar are taken and compared with Wet Cracks standards, as follows: Grade I means no crack or only hair crack at the flash; Grade II means slight crack at the side joins; Grade III means wide crack at the side joins including latitude and longitude; Grade IV means wider crack over the whole join; Grade V means open crack over the whole join and other surface.

Transparency Test Method:

The clarity of the bars was evaluated using percent transmittance by placing a 1 cm thick sample of the bar in the beam of a Cary 100 UV-Visible Spectrophometer at a wavelength of 800 nm. Within the context of this invention, a bar is deemed to be transparent (clear) if the maximum transmittance of light at a wavelength of 800 nm through a 1 cm sample is at least 35%, preferably at least 50%. The bar is deemed translucent if the maximum transmittance of such light through the sample is between 2% and less than 35%. A bar is deemed opaque if the maximum transmittance of such a light is less than 2%.

Hardness Test Method:

The hardness measurement correlates to the maximum force in Newtons (N) for a cylinder probe to penetrate the solid composition for a set depth of 8 mm using a TA.XTPlus Texture Analyzer with a 0.5 inch (1.27 cm) Radius Cylinder (P/0.5R) cylinder probe. The test method was used with the following settings: test mode is compression, pre-test speed is 0.5 mm/sec, test speed is 0.5 mm/sec, post-test speed is 0.5 mm/sec, target mode is distance, trigger type is force, and trigger force is 0.0493N. Three measurements of the force were taken and the average of the three measurements was reported.

Foaming Test Method:

The Foam Volume in centimeters (cm) was measured in accord with the Ross-Mile test method (ISO696-1975 or GB7462-87) at a soap concentration of 0.4 wt. % and a water harness of 150 ppm.

pH Value:

The pH of the solid compositions was tested as a 10% solution of the compositions in water at 25° C.

“Sweat” Test:

The solid compositions in bar form were subjected to a sweating test which was designed to mimic the humid atmosphere conditions frequently found in modern bathrooms where poor ventilation in combination with the use of hot water can produce a high relative humidity. The test employed comprised storing the bars under ambient conditions with a relative humidity of 65% and examining visually the products daily for evidence of sweating. The presence of sweat was scored on a ten point scale, 0 signifying absence of sweat and 10 signifying a bar coated with a wet layer.

Gelling Point:

After addition and mixture of all ingredients of a composition, the heated homogenous liquid is cooled and begins to solidify at a certain temperature, which temperature is recorded as the gelling point.

### Example 1

Several solid compositions of the present invention (E1-4) and two comparative compositions (C1-C2) were made and tested in accord with the following procedure.

Tripropylene glycol methyl ether (TPM) or propylene glycol (PG) was charged for each composition in the amount indicated in Table 1 into a 1 kg vessel and heated to 80° C. Dibenzylidene sorbitol (1.0 g) and hydroxypropylmethylcellulose (1.0 g) were sprinkled into the heated batch. The temperature was then raised to 120-130° C. with mixing until clear mucilage was formed. Water, in the amount indicated in Table 1, was added into the heated batch and mixing was continued until a homogenous mass was obtained. The batch was cooled to about 80° C. and then poured in a plastic mould which was resistant to 80° C. hot pour temperature. The solid gel was allowed to cool and harden at ambient conditions (approximately 5-8 hours). The hardness and transparency for each composition was measured using the Hardness Test Method and reported in Table 1.

TABLE 1

| Composition | E1 | E2 | E3 | E4 | C1 | C2 |
|---|---|---|---|---|---|---|
| TPM (wt. %) | 58 | 48 | 38 | 28 | 0 | 0 |
| PG (wt. %) | 0 | 0 | 0 | 0 | 38 | 28 |
| Water (wt. %) | 40 | 50 | 60 | 70 | 60 | 70 |
| Hardness | 18.80 | 17.11 | 17.11 | 7.98 | Did not | Did not |
| | | | | | solidify | solidify |

### Example 2

Several solid compositions of the present invention (E5-E12) and two comparative compositions (C3-C4) were made and tested in accord with the following procedure.

Tripropylene glycol methyl ether (TPM), Dipropylene glycol methyl ether (DPM), propylene glycol (PG), or a combination thereof was charged for each composition in the weight percent amounts indicated in Table 2 into a 1 kg vessel and heated to 80° C. The gelling agent, e.g. dibenzylidene sorbitol (DBS), dibutyl lauroyl glutamate (GP-1), or stearic acid (SA) and sodium hydroxide (NaOH), and solidifying synergist, e.g. hydroxypropylmethylcellulose (HPMC), if any, were sprinkled into the heated batch in the weight percent amounts indicated in Table 2 for each composition. The temperature was then raised to 120-130° C. with mixing until clear mucilage was formed. Water, in the weight percent amount indicated in Table 2, was added into the heated batch and mixing was continued until a homogenous mass was obtained. The batch was cooled to about 80° C. and then poured in a plastic mould which was resistant to 80° C. hot pour temperature. The solid gel was allowed to cool and harden at ambient conditions (about 27° C. for approximately 5-8 hours). The approximate gelling point for all compositions was measured and reported in Table 2.

TABLE 2

| | E5 | E6 | C3 | E7 | E8 | E9 | E10 | E11 | E12 | C4 |
|---|---|---|---|---|---|---|---|---|---|---|
| TPM | 58 | 0 | 0 | 49 | 0 | 40 | 20 | 75 | 45 | 0 |
| DPM | 0 | 58 | 0 | 0 | 49 | 0 | 0 | 0 | 0 | 0 |
| PG | 0 | 0 | 58 | 0 | 0 | 0 | 20 | 0 | 0 | 45 |
| DBS | 1.5 | 1.5 | 1.5 | 1.5 | 1.5 | 1.5 | 1.5 | 0 | 0 | 0 |
| GP-1 | 0 | 0 | 0 | 0 | 0 | 0 | 0 | 5 | 0 | 0 |
| SA | 0 | 0 | 0 | 0 | 0 | 0 | 0 | 0 | 20 | 20 |
| NaOH | 0 | 0 | 0 | 0 | 0 | 0 | 0 | 0 | 4 | 4 |
| HPMC | 0.5 | 0.5 | 0.5 | 0.5 | 0.5 | 0.5 | 0.5 | 0 | 0 | 0 |
| Water | 40 | 40 | 40 | 49 | 49 | 58 | 58 | 20 | 31 | 31 |
| Gel point | 55-60 | 55-60 | 90-95 | 65-70 | 65-70 | 65-70 | 95-100 | 90 | 60-65 | 70-75 |
| (° C.) | | | | | | | | | | |

### Example 3

Several solid compositions of the present invention (E13-20) were made in accord with the following procedure.

Tripropylene glycol methyl ether (TPM), and where indicated, one or more co-solvents (e.g. propylene glycol (PG), 1,3-butylene glycol (1,3-BG), glycerin (Gly), or a combination of two thereof) were charged for each composition in the weight percent amounts indicated in Table 3 into a 1 kg vessel and heated to 80° C. Dibenzylidene sorbitol (DBS) and hydroxypropylmethylcellulose (HPMC) were sprinked into the heated batch in the weight percent amounts indicated in Table 3 for each composition. The temperature was then raised to 120-130° C. with mixing until clear mucilage was formed. Any surfactants or other ingredients as indicated in Table 3 (e.g. cocamidopropyl betaine (CAPB), coco-glucoside (CG), sodium laureth sulfate-2EO (SLES), sodium C12-16 olefin sulfonate (AOS35), sodium lauroamphoacetate (SLA), Cocamide MEA (CMEA), sodium lauroyl glutamate (SLG), sodium cocoyl isethionate (SCl), sodium cocoyl glutamate (SCG), disodium laureth sulfosuccinate (DLS), PEG-150 Distearate (PEG150), urea) were added into the heated batch followed by the addition of water under stirring to get a homogenous mixture. The batch was cooled to about 80° C. and then poured in a plastic mould which was resistant to 80° C. hot pour temperature and shaped to produce solid compositions that were cylinder shape 15-20 mm high by 60 mm diameter, approximately 50˜80 g. The solid gel was allowed to cool and harden at ambient conditions (approximately 5-8 hours).

Applicants note the weight percent amounts of the liquid surfactants in Table 3 are weight percents of the commercial surfactant product, i.e. in E13, the 6.0 wt. % of CAPB is 6.0 weight percent of the Evonik product available as TEGO® Betain F 50J (see above) which is approximately 44% active solids and 56% water. Accordingly, the active solids amount of CAPB in E13 is 44% of 6.0 or 2.64 active weight percent. The total weight percent of water in each of the compositions is the weight percent of water into which the surfactants are added (the last row of Table 3) plus any water added with the commercial surfactants or other products. For example, in E13, the total weight percent of water in the composition includes the 20 wt. % into which the surfactants were added, plus the additional water from the CAPB (3.36 wt. %) and the CG (8.0 wt. %) for a total weight percent of water in E13 of 31.36 wt. %

TABLE 3

| | E13 | E14 | E15 | E16 | C5 | E17 | E18 | E19 | E20 |
|---|---|---|---|---|---|---|---|---|---|
| TPM | 26.0 | 5.0 | 10.0 | 5.0 | 0 | 40.0 | 28.0 | 28.0 | 5.0 |
| 1,3-BG | 30.0 | 25.0 | 20.0 | 25.0 | 25.0 | 0 | 0 | 10.0 | 25.0 |
| PG | 0 | 28.0 | 28.0 | 28.0 | 28.0 | 0 | 25.0 | 0 | 27.5 |
| Gly | 0 | 0 | 0 | 0 | 5.0 | 0 | 5.0 | 12.0 | 0 |
| DBS | 1.5 | 1.5 | 1.5 | 1.5 | 1.5 | 2.0 | 2.0 | 1.5 | 1.5 |
| HPMC | 0.5 | 0.5 | 0.5 | 0.5 | 0.5 | 2.0 | 2.0 | 0.5 | 0.5 |
| CAPB | 6.0 | 3.0 | 0 | 0 | 0 | 6.0 | 5.4 | 0 | 0 |
| CG | 16.0 | 0 | 0 | 0 | 0 | 14.0 | 0 | 0 | 0 |
| SLES | 0 | 9.0 | 0 | 0 | 0 | 0 | 13.6 | 0 | 0 |
| AOS35 | 0 | 6.0 | 0 | 0 | 0 | 0 | 6.0 | 0 | 0 |
| SLA | 0 | 2.0 | 0 | 0 | 0 | 0 | 2.0 | 0 | 0 |
| CMEA | 0 | 1.3 | 0 | 0 | 0 | 0 | 1.0 | 0 | 0 |
| SLG | 0 | 0 | 20.0 | 0 | 0 | 0 | 0 | 0 | 0 |
| SCI | 0 | 0 | 0 | 25.0 | 0 | 0 | 0 | 20.0 | 0 |
| SCG | 0 | 0 | 0 | 0 | 15.0 | 0 | 0 | 0 | 0 |
| DLS | 0 | 0 | 0 | 0 | 0 | 0 | 0 | 0 | 25.0 |
| PEG150 | 0 | 0 | 0 | 0 | 0 | 0 | 0 | 0 | 0.5 |
| urea | 0 | 0 | 0 | 0 | 0 | 6.0 | 0 | 8.0 | 0 |
| water | 20.0 | 18.7 | 20.0 | 15.0 | 25.0 | 30.0 | 10.0 | 20.0 | 15.0 |

### Example 4

The mush, crack, pH, foam volume, and sweat for compositions in Table 3 were measured using the respective test methods described above. In addition, soap bars were made in accord with Example of 1 of U.S. Pat. No. 6,514,919 (C6) and Example 1 of U.S. Pat. No. 7,045,491 (C7) and tested in accord with the test methods. Also, three commercially available products were tested in accord with the test methods: (1) Johnson's® Baby soap bar (125 g) having an ingredient listing of: Sodium Palmitate, Sodium Oleate, Water, Sodium Laurate, Sodium Stearate, Sodium Linoleate, Sodium Myristate, Glycerin, Titanium Dioxide (CI 77891), Tetrasodium EDTA, Phosphoric acid, Etidronic acid, Fragrance, Avena Sativa (Oat) Kernel Flour (JBS); (2) Dove which bar/what size (Dove White Beauty Bar, 100 g) having an ingredient listing of: Sodium Cocoyl Isethionate, Stearic Acid, Coconut Acid, Sodium Tallowate, Water, Sodium Isethionate, Sodium Stearate, Cocamidopropyl Betaine, Sodium Cocoate, Palm Kernelate, Fragrance (Parfum), Sodium Chloride, Tetrasodium EDTA, Trisodium Etidronate, BHT, Titanium Dioxide (CI 77891), Sodium Dodecylbenzene Sulfonate (DOVE); and (3) Neutrogena (Original formula, 100 g) soap bar having an ingredient listing of: TEA Stearate, Triethanolamine, Sodium Tallowate, Glycerin, Water Purified, Sodium Cocoate, Sodium Ricinoleate, TEA Oleate, Cocamide DEA, Tocopherol (NSB). The results of the testing are shown in Table 4.

TABLE 4

| | Mush | | | Foam Vol. | |
|---|---|---|---|---|---|
| | (g/50 cm²) | Crack | pH | (cm) | Sweat |
| E14 | No Mush | No Crack | 6.88 | 17.5 | Not tested |
| E15 | No Mush | No Crack | 7.80 | 21.0 | Not tested |
| E16 | No Mush | No Crack | 5.77 | 16.5 | Not tested |
| E17 | No Mush | No Crack | 4.80 | 18.5 | Not tested |
| C5 | No Mush | No Crack | 5.57 | 20.5 | Not tested |
| E18 | No Mush | No Crack | 6.84 | 14.0 | 2 |
| E19 | No Mush | No Crack | 6.80 | 21.0 | 0 |
| E20 | No Mush | No Crack | 6.88 | 19.0 | 0 |
| E21 | No Mush | No Crack | 5.52 | 8.5 | 1 |
| C6 | 2.1 | Too soft | 7.42 | 15.0 | 10 |
| C7 | 16.19 | Grade II | 8.56 | 6.0 | 0 |
| JBS | 8.5 | Grade II | 10.21 | 21.4 | 0 |
| DOVE | 4.9 | Grade II | 7.05 | 10.2 | 0 |
| NSB | 10.2 | Too soft | 9.18 | 14.3 | 9 |

### Example 5

The clarity for compositions E1-E20, C1-C7, JBS, DOVE, and NSB from the previous examples were measured using the Transparency Test. Each sample was evaluated with a minimum of two readings. The results are shown in Table 5.

TABLE 5

| Sample no. | Average % Transmittance at 800 nm | Clarity |
|---|---|---|
| E1 | 72.62% | Transparent |
| E2 | 69.34% | Transparent |
| E3 | 64.34% | Transparent |
| E4 | 47.48% | Transparent |
| E5 | 76.25% | Transparent |
| E6 | 40.45% | Transparent |
| C3 | 27.17% | Translucent |
| E7 | 70.56% | Transparent |
| E8 | 40.23% | Transparent |
| E9 | 62.58% | Transparent |
| E10 | 37.35% | Transparent |
| E11 | 1.23% | Opaque |
| E12 | 10.57% | Translucent |
| C4 | 2.42% | Translucent |
| E13 | 48.00% | Transparent |
| E14 | 39.58% | Transparent |
| E15 | 0.27% | Opaque |
| E16 | 0.021% | Opaque |
| C5 | 5.36% | Translucent |
| E17 | 55.63% | Transparent |
| E18 | 40.50% | Transparent |
| E19 | 0.022% | Opaque |
| E20 | 44.37% | Transparent |
| C6 | 45.56% | Transparent |
| C7 | 1.33% | Opaque |
| JBS | 0.001% | Opaque |
| DOVE | 0.001% | Opaque |
| NSB | 72.24% | Transparent |

### Example 6

Solid compositions (E21-E22) were made from commercially available liquid compositions in accord with the present invention. The compositions were prepared by adding the commercial liquid shower compositions: (1) Safeguard Aloe Pink liquid shower gel from P&G having an ingredient listing of Water, Sodium Laureth Ether Sulfate, Sodium Chloride, Sodium Laureth Sulfate, Cocamidopropyl Betaine, Fragrance, Lauric Acid, Sodium Citrate, Acrylates Copolymer, Sodium Benzoate, Disodium EDTA, Polyquaternium 7, Extract Aloe Vera, Triclocarban, Methylchloroisothiazolinone, Methylisothiazolinone; or (2) Palmolive Caprice Naturals Clear Red liquid shampoo from Colgate having an ingredient listing of Water, Ammonium Laureth Sulfate, Ammonium Lauryl Sulfate, Cocamidopropyl Betaine, Cocamide DEA, Fragrance, DMDMH, Critic Acid, Extract (pyus malus), Tetrasodium EDTA, CI 16035, Methylchloroisothiazolinone, Methylisothiazolinone into a heated mixture of tripropylene glycol methyl ether, dibenzylidene sorbitol and hydroxypropyl methyl cellulose in the weight percent amounts shown in Table 6, and mixing to obtain a homogenous composition. This homogenous composition was then poured into suitable molds of different forms made of plastic or rubber and allowed to cool and harden at ambient conditions. The hardness and appearance were evaluated and the results indicated in Table 6.

TABLE 6

| | E21 | E22 |
|---|---|---|
| TPM | 38 | 46 |
| DBS | 1.0 | 2.0 |
| HPMC | 1.0 | 2.0 |
| Safeguard Aloe Pink liquid | 60 | 0 |
| shower gel | | |
| Palmolive Caprice Naturals | 0 | 50 |
| Clear Red liquid shampoo | | |
| Hardness (N) | 8.68 | 34.46 |
| Appearance | white, opaque | red, transparent |

### Example 7

Several solid compositions of the present invention (E23-E26) were made in accord with the procedure and formulation for E14 except that an additional solidifying synergist is added in the amount listed in Table 7 (and an equal amount of water is removed to keep the remainder of the weight percents in E14 the same).

TABLE 7

| | Solidifying synergist | Wt. % |
|---|---|---|
| E23 | PEG-150 distearate | 2.0 |
| E24 | PEG-120 Methyl Glucose | 2.0 |
| | Dioleate | |
| E25 | Gellan gum | 0.5 |
| E26 | Glyceryl oleate citrate | 0.05 |

What is claimed is:

1. A solid composition comprising a glycol ether, a gelling agent, and water.

2. The composition of claim 1 wherein said composition has a Total Mush according to the Mush Test Method of about 5 g/cm² or less.

3. The composition of claim 1 wherein said composition has a Total Mush according to the Mush Test Method of about 1 g/cm² or less.

4. The composition of claim 1 wherein said glycol ether is selected from the group consisting of ethylene glycol alkyl ethers, ethylene glycol aryl ethers, ethylene glycol dialkyl ethers, diethylene glycol alkyl ethers, dipropylene glycol alkyl ethers, tripropylene glycol alkyl ethers, and combinations of two or more thereof.

5. The composition of claim 4 wherein said glycol ether comprises dipropylene glycol methyl ether, tripropylene glycol methyl ether, or a combination thereof.

6. The composition of claim 1 wherein said gelling agent is selected from the group consisting of dibenzylidene sorbitol, dibenzylidene xylitol, dibenzylidene ribitol, 4-methyl dibenzylidene sorbitol, 4-ethyl dibenzylidene sorbitol, 4-chloro dibenzylidene sorbitol, 2,4-dimethyl dibenzylidene sorbitol, bis(3,4-dimethylesbenzylidene) sorbitol acetal, tribenzylidene sorbitol and mixtures of two or more thereof.

7. The composition of claim 1 wherein said gelling agent comprises dibenzylidene sorbitol.

8. The composition of claim 1 wherein said composition is essentially free of metal soaps.

9. The composition of claim 1 comprising from about 10% to about 70% water.

10. The composition of claim 1 comprising at least 35% by weight of water.

11. The composition of claim 1 further comprising a solidifying synergist.

12. The composition of claim 11 wherein said solidifying synergist is selected from the group consisting of hydroxypropyl cellulose, polyethylene glycol esters, gellan gums, guar gums, and the like.

13. The composition of claim 12 wherein said solidifying synergist comprises hydroxypropyl methyl cellulose.

14. The composition of claim 1 further comprising a co-solvent.

15. The composition of claim 14 wherein said co-solvent comprises glycol, glycerin, 1,3-butylene glycol, sorbitol, or a combination of two or more thereof.

16. The composition of claim 14 wherein said composition is essentially free of glycerin.

17. The composition of claim 1 further comprising surfactant selected from the group consisting of anionic surfactants, amphoteric surfactants, nonionic surfactants, cationic surfactants and combinations of two or more thereof.

18. The composition of claim 17 wherein said surfactant comprises at least one anionic surfactant.

19. The composition of claim 17 wherein said surfactant is a liquid surfactant.

20. The composition of claim 19 wherein said composition is transparent.

21. The composition of claim 1 comprising from about 5 to about 75% of glycol ether comprising dipropylene glycol methyl ether, tripropylene glycol methyl ether, or a combination thereof, from about 0.5 to about 5% of gelling agent comprising dibenzylidene sorbitol, from about 10 to about 70% of water, and further comprising at least one surfactant and from about 0.05 to about 5% of hydroxypropyl methyl cellulose.

22. The composition of claim 21 further comprising at least one co-solvent selected from the group consisting of glycol, 1,3-butylene glycol, sorbitol, or a combination of two or more thereof.

23. The composition of claim 21 wherein said composition is a cleansing bar.

24. A method of making a solid composition comprising combining a heated gelling mixture comprising glycol ether and a gelling agent with an aqueous liquid and cooling to form a solid composition.

25. The method of claim 24 wherein said heated gelling mixture is heated to a temperature of from about 80° C. to about 140° C. prior to addition of the aqueous liquid.

26. The method of claim 24 wherein said glycol ether comprises dipropylene glycol methyl ether, tripropylene glycol methyl ether, or a combination thereof.

27. The method of claim 24 wherein said gelling agent is selected from the group consisting of dibenzylidene sorbitol, dibenzylidene xylitol, dibenzylidene ribitol, 4-methyl dibenzylidene sorbitol, 4-ethyl dibenzylidene sorbitol, 4-chloro dibenzylidene sorbitol, 2,4-dimethyl dibenzylidene sorbitol, bis(3,4-dimethylesbenzylidene) sorbitol acetal, tribenzylidene sorbitol and mixtures of two or more thereof.

28. The method of claim 24 wherein said heated gelling mixture further comprises a solidifying synergist selected from selected from the group consisting of hydroxypropyl cellulose, polyethylene glycol esters, gellan gums, guar gums, and the like

29. The method of claim 24 wherein said heated gelling mixture further comprises a co-solvent selected from the group consisting of glycol, 1,3-butylene glycol, sorbitol, or a combination of two or more thereof.

30. The method of claim 24 wherein said aqueous liquid further comprises at least one surfactant.
